SEQUENCE LISTING (31 sequences)

SEQ ID NO: 1 - protein, 16 aa, Artificial Sequence
  Targeted aggregate peptide
AEAEAKAKAEAEAKAK

SEQ ID NO: 2 - protein, 8 aa, Artificial Sequence
  Targeted aggregate peptide
MDYEIKFH

SEQ ID NO: 3 - protein, 8 aa, Artificial Sequence
  Targeted aggregate peptide
MDYNIQFH

SEQ ID NO: 4 - protein, 8 aa, Artificial Sequence
  Targeted aggregate peptide
MDYKFKFN

SEQ ID NO: 5 - protein, 7 aa, Artificial Sequence
  Targeted aggregate peptide
NFDLNLD

SEQ ID NO: 6 - protein, 7 aa, Artificial Sequence
  Targeted aggregate peptide
EIQFEID

SEQ ID NO: 7 - protein, 7 aa, Artificial Sequence
  Targeted aggregate peptide
NIDFQFD

SEQ ID NO: 8 - protein, 7 aa, Artificial Sequence
  Targeted aggregate peptide
DLQLQIR

SEQ ID NO: 9 - protein, 7 aa, Artificial Sequence
  Targeted aggregate peptide
DIEIEIR

SEQ ID NO: 10 - protein, 7 aa, Artificial Sequence
  Targeted aggregate peptide
EVDIEIR

SEQ ID NO: 11 - protein, 7 aa, Artificial Sequence
  Targeted aggregate peptide
RVQVHIH

SEQ ID NO: 12 - protein, 7 aa, Artificial Sequence
  Targeted aggregate peptide
RVHIQLN

SEQ ID NO: 13 - protein, 7 aa, Artificial Sequence
  Targeted aggregate peptide
RVHINLD

SEQ ID NO: 14 - protein, 7 aa, Artificial Sequence
  Targeted aggregate peptide
KVDFHVN

SEQ ID NO: 15 - protein, 7 aa, Artificial Sequence
  Targeted aggregate peptide
HIKVDFH

SEQ ID NO: 16 - protein, 7 aa, Artificial Sequence
  Targeted aggregate peptide
QLKFHVN

SEQ ID NO: 17 - protein, 7 aa, Artificial Sequence
  Targeted aggregate peptide
DVEVKMH

SEQ ID NO: 18 - protein, 7 aa, Artificial Sequence
  Targeted aggregate peptide
ELQIDMH

SEQ ID NO: 19 - protein, 7 aa, Artificial Sequence
  Targeted aggregate peptide
EFNLKMH

SEQ ID NO: 20 - protein, 14 aa, Artificial Sequence
  Targeted aggregate peptide
DLENLLEKFEQLIK

SEQ ID NO: 21 - protein, 14 aa, Artificial Sequence
  Targeted aggregate peptide
KLNHVVQELQELVQ

SEQ ID NO: 22 - protein, 14 aa, Artificial Sequence
  Targeted aggregate peptide
KLKNLLNDFEDLIN

SEQ ID NO: 23 - protein, 14 aa, Artificial Sequence
  Targeted aggregate peptide
NVQQLLKKLQQMIQ

SEQ ID NO: 24 - protein, 14 aa, Artificial Sequence
  Targeted aggregate peptide
EIEDLLQKLQELME

SEQ ID NO: 25 - protein, 14 aa, Artificial Sequence
  Targeted aggregate peptide
KIQKIIEKVNELMQ

SEQ ID NO: 26 - protein, 14 aa, Artificial Sequence
  Targeted aggregate peptide
DLHNLINKLDDVMQ

SEQ ID NO: 27 - protein, 14 aa, Artificial Sequence
  Targeted aggregate peptide
KMHDLIDDLHHLLN

SEQ ID NO: 28 - protein, 14 aa, Artificial Sequence
  Targeted aggregate peptide
KLNDLLEDLQEVLK

SEQ ID NO: 29 - protein, 14 aa, Artificial Sequence
  Targeted aggregate peptide
HLQNVIEDIHDFMQ

SEQ ID NO: 30 - protein, 14 aa, Artificial Sequence
  Targeted aggregate peptide
KLQEMMKEFQQVLD

SEQ ID NO: 31 - protein, 14 aa, Artificial Sequence
  Targeted aggregate peptide
NIKEIFHHLEELVH

(19) United States
(12) Patent Application Publication
(10) Pub. No.: US 2004/0204561 A1
(43) Pub. Date: Oct. 14, 2004

(54) Peptide aggregates

(76) Inventor: Jeffrey J. Ellison, (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/635,838

(22) Filed: Aug. 6, 2003

Related U.S. Application Data
(60) Provisional application No. 60/401,617, filed on Aug. 6, 2002.

(51) Int. Cl. C07K 7/08; C07K 7/06

(52) U.S. Cl. 530/326; 530/327; 530/328; 530/329; 530/400

(57) ABSTRACT

Disclosed are peptide aggregates that include assembling peptides optionally linked to metal binding moieties and/or target binding moieties. Also disclosed are methods of using such peptide aggregates for magnetic resonance imaging.

### RELATED APPLICATION DATA

This application claims priority from U.S. Provisional Application Serial No. 60/401,617, filed Aug. 6, 2002.

### TECHNICAL FIELD

This invention relates to assembling peptides and peptide aggregates useful for magnetic resonance imaging.

### BACKGROUND

Magnetic resonance (MR) imaging of low abundance (e.g., 1×10⁻⁶M to 1×10⁻⁹M) biological targets is limited by the signal generating capacity of the imaging agent employed. One way to image low abundance biological targets involves using macromolecular conjugates (e.g., conjugates of dendrimers, synthetic polymers, proteins, and polysaccharides) that have multiple paramagnetic moieties. Such macromolecular conjugates can be difficult to synthesize, and can be distributed only to the plasma compartment where they may exhibit long plasma half lives, long elimination half lives, and incomplete elimination.

Another way to image low abundance biological targets involves associating large numbers of paramagnetic moieties via micelles and liposomes. Micelles and liposomes, however, sequester associated paramagnetic moieties away from bulk water, limiting their diagnostic utility. In addition, the general lipophilicity and the high mass percentage of micellar or liposomal material required to associate paramagnetic moieties can cause toxicity problems for the individual under study.

### SUMMARY

The invention features peptide aggregates and assembling peptides useful for magnetic resonance imaging. Peptide aggregates in accord with the invention include individual (monomeric) assembling peptides. An assembling peptide can include, by covalent attachment, one or more metal binding moieties. Peptide aggregates that include assembling peptides linked to metal binding moieties may be referred to as metallopeptide aggregates and are useful as MR contrast agents. Metallopeptide aggregates can have multiple paramagnetic metal centers and exhibit increased size, increased solubility, and higher MR relaxivity relative to the individual monomeric assembling peptides from which they are assembled. In addition, such aggregates are peptide-based and can be cleared rapidly from a subject under study by in vivo enzymatic degradation of the peptide portion of the molecule.

Assembling peptides also can be linked covalently to one or more target binding moieties that exhibit affinity for a particular target (e.g., a biological target such as a polypeptide, enzyme, receptor, nucleic acid (e.g., DNA or RNA), and tissue). Peptide aggregates that include an assembling peptide linked to a target binding moiety are referred to as “targeted” peptide aggregates. Targeted peptide aggregates can bind targets, including preselected biological targets.

The invention features peptide aggregates that can include assembling peptides that are linked covalently to neither of, either of, or both of a metal binding moiety and a target binding moiety. For example, some assembling peptides contain both a target binding moiety and a metal binding moiety. Other assembling peptides contain a target binding moiety. Yet other assembling peptides contain a metal binding moiety. Typically, the featured peptide aggregates include assembling peptides at least some of which contain a metal binding moiety. In addition, featured peptide aggregates typically include assembling peptides at least some of which contain a target binding moiety. In some embodiments, a peptide aggregate has a hydrodynamic radius of 2 nm to 500 nm. In some embodiments, a peptide aggregate includes assembling peptides that comprise a target binding moiety having affinity for a target. In some embodiments, a peptide aggregate includes assembling polypeptides that contain a metal binding moiety and a target binding moiety having affinity for a target. In other embodiments, a peptide aggregate includes some assembling peptides that have hydrophilic amino acids that are only positively charged, and other assembling peptides have hydrophilic amino acids that are only negatively charged. An assembling peptide can be a self-assembling peptide; e.g., a peptide that can associate with itself. Any of the peptide aggregates described herein can include self-assembling peptides.

Also featured are methods for magnetic resonance imaging. The method involves introducing a peptide aggregate or assembling peptide according to the invention to a subject (e.g., a mammal such as a human), and subjecting the subject to magnetic resonance imaging.

Other features and advantages of the invention will be apparent from the following detailed description, and from the claims. The disclosed materials, methods, and examples are illustrative only and are not intended to be limiting. Skilled artisans will appreciate that methods and materials similar or equivalent to those described herein can be used to practice the invention.

Unless otherwise defined, all technical and scientific terms used herein have the meaning commonly understood by one skilled in the art to which this invention belongs. All publications, patent applications, patents, and other references mentioned herein are incorporated by reference in their entirety. In case of conflict, the present specification, including definitions, will control.

### DESCRIPTION OF DRAWINGS

FIG. 1 is a schematic of a metallopeptide aggregate.

FIG. 2 is a schematic of a targeted metallopeptide aggregate.

FIG. 3 illustrates three structures of self-assembling peptides comprising metal binding moieties.

FIG. 4 demonstrates the relationship of relaxivity vs. molecular weight for a peptide aggregate of the invention.

FIG. 5 demonstrates SEC-LS measurements of peptide aggregates of the invention.

FIG. 6 is a CD spectrum for a peptide aggregate of the invention.

FIG. 7 sets forth two structures of self-assembling peptides comprising target binding moieties.

FIG. 8 illustrates the percent capture of various peptide aggregates of the invention by streptavidin-coated beads.

### DETAILED DESCRIPTION

Definitions

Commonly used chemical abbreviations that are not explicitly defined in this disclosure may be found in The American Chemical Society Style Guide, Second Edition; American Chemical Society, Washington, D.C. (1997), “2001 Guidelines for Authors” J. Org. Chem. 66(1), 24A (2001), “A Short Guide to Abbreviations and Their Use in Peptide Science” J. Peptide. Sci. 5, 465-471 (1999).

The terms “chelating ligand,” “chelating moiety,” and “chelate moiety” may be used to refer to any polydentate ligand which is capable of coordinating a metal ion, including DTPA (and DTPE), DOTA, DOTAGA, DO3A, or NOTA molecule, or any other suitable polydentate chelating ligand as is further defined herein, that is either coordinating a metal ion or is capable of doing so, either directly or after removal of protecting groups, or is a reagent, with or without suitable protecting groups, that is used in the synthesis of a contrast agent and comprises substantially all of the atoms that ultimately will coordinate the metal ion of the final metal complex. The term “chelate” refers to the actual metal-ligand complex, and it is understood that the polydentate ligand will typically be coordinated to a medically useful metal ion.

The term “affinity” as used herein refers to the capacity of a peptide aggregate to be taken up by, retained by, or bound to a particular target, e.g., a biological target, to a greater degree than other components. Peptide aggregates that have this property are said to be “targeted” to the “target” component. Aggregates that lack this property are said to be “non-specific” or “non-targeted.” The binding affinity of a target binding moiety for a target may be expressed in terms of the equilibrium dissociation constant “Kd.”

For the purposes of this application, “DTPA” refers to a chemical compound comprising a substructure composed of diethylenetriamine, wherein the two primary amines are each covalently attached to two acetyl groups and the secondary amine has one acetyl group covalently attached according to the following formula:

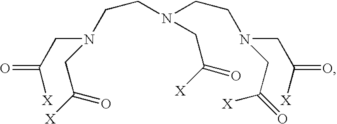

wherein X is a heteroatom electron-donating group capable of coordinating a metal cation, preferably O⁻, OH, NH₂, OPO₃²⁻, or NHR, or OR wherein R is any aliphatic group. When each X group is tert-butoxy (tBu), the structure may be referred to as “DTPE” (“E” for ester).

For the purposes of this application, “DOTA” refers to a chemical compound comprising a substructure composed of 1,4,7,11-tetraazacyclododecane, wherein the amines each have one acetyl group covalently attached according to the following formula:

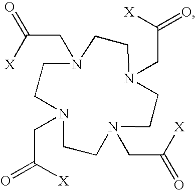

wherein X is defined above.

For the purposes of this application, “NOTA” refers to a chemical compound comprising a substructure composed of 1,4,7-triazacyclononane, wherein the amines each have one acetyl group covalently attached according to the following formula:

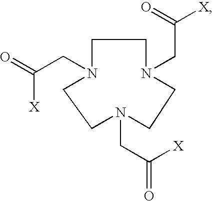

wherein X is defined above.

For the purposes of this application, “DO3A” refers to a chemical compound comprising a substructure composed of 1,4,7,11-tetraazacyclododecane, wherein three of the four amines each have one acetyl group covalently attached and the other amine has a substituent having neutral charge according to the following formula:

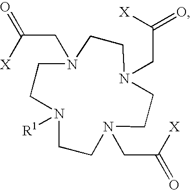

wherein X is defined above and R¹ is an uncharged chemical moiety, preferably hydrogen, any aliphatic, alkyl group, or cycloalkyl group, and uncharged derivatives thereof. The preferred chelate “HP”-DO3A has R¹═—CH₂(CHOH)CH₃.

For the purposes of this application, “DOTAGA” refers to a chemical compound comprising a substructure composed of 1,4,7,11-tetraazacyclododecane and having the structure (shown complexed to Gd(III)):

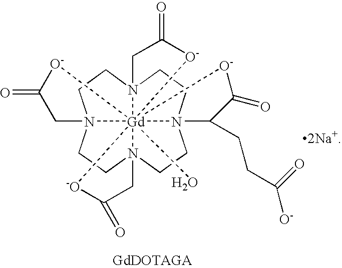

In each of the structures above, the carbon atoms of the ethylene groups may be referred to as “backbone” carbons. The designation “bbDTPA” may be used to refer to the location of a chemical bond to a DTPA molecule (“bb” for “back bone”). Note that as used herein, bb(CO)DTPA means a C═O moiety bound to an ethylene backbone carbon atom of DTPA.

As used herein, the term “purified” refers to a peptide that has been separated from either naturally occurring organic molecules with which it normally associates or, for a chemically-synthesized peptide, separated from any other organic molecules present in the chemical synthesis. Typically, the polypeptide is considered “purified” when it is at least 70% (e.g., 70%, 80%, 90%, 95%, or 99%), by dry weight, free from any other proteins or organic molecules.

As used herein, the term “peptide” refers to a chain of amino acids that is about 2 to about 50 amino acids in length (e.g., 10 to 30 amino acids in length).

As used herein, the term “natural” or “naturally occurring” amino acid refers to one of the twenty most common occurring amino acids. Natural amino acids modified to provide a label for detection purposes (e.g., radioactive labels, optical labels, or dyes) are considered to be natural amino acids. Natural amino acids are referred to by their standard one- or three-letter abbreviations.

The term “non natural amino acid” or “non-natural” refers to any derivative of a natural amino acid including D forms, β and γ amino acid derivatives, or other derivatives. It is noted that certain amino acids, e.g., hydroxyproline, may be found in nature within a certain organism or a particular protein.

The term “relaxivity” as used herein refers to the increase in either of the MRI quantities 1/T1 or 1/T2 per millimolar (mM) concentration of paramagnetic ion or contrast agent, wherein T1 is the longitudinal or spin-lattice, relaxation time, and T2 is the transverse or spin-spin relaxation time of water protons or other imaging or spectroscopic nuclei, including protons found in molecules other than water. Relaxivity is expressed in units of mM⁻¹s⁻¹.

The terms “target binding,” “binding,” or “biological target binding” are used interchangeably and refer to non-covalent interactions of a peptide aggregate or assembling peptide described herein with a target. These non-covalent interactions are independent from one another and may be, inter alia, hydrophobic, hydrophilic, dipole-dipole, pi-stacking, hydrogen bonding, electrostatic associations, or Lewis acid-base interactions.

The term “assembling peptide” means a peptide that has the ability to associate noncovalently with another peptide, which can have the same or different amino acid sequence, to form a peptide aggregate. A “peptide aggregate” is a noncovalent association of two or more assembling peptides. A “metallopeptide aggregate” is a peptide aggregate containing at least one assembling peptide comprising a metal binding moiety. A “targeted peptide aggregate” is a peptide aggregate containing at least one assembling peptide comprising a target binding moiety. A “targeted metallopeptide aggregate” is a peptide aggregate containing at least one assembling peptide comprising a metal binding moiety and at least one assembling peptide comprising a target binding moiety.

Assembling Peptides

The invention provides peptide aggregates that include assembling peptides. Assembling peptides are typically 2 to 50 amino acids in length, e.g., 8, 9, 10, 11, 12, 13, 14, 15, 16, 17, 18, 19, 20, 25, 30, 35, 40, 45 or 50 amino acids in length. Assembling peptides are typically purified peptides. Assembling peptides can be synthesized and purified by a number of methods known to those of skill in the art, e.g., solid phase synthesis methods such as those disclosed in WO 01/09188 or in WO 01/08712.

Suitable amino acids include natural and non-natural amino acids. Amino acids with many different protecting groups appropriate for immediate use in the solid phase synthesis of peptides are commercially available. In addition to the twenty most common naturally occurring amino acids, the following non-natural amino acids or amino acid derivatives may be used: β-Alanine (β-Ala), γ-Aminobutyric Acid (GABA), 2-Aminobutyric Acid (2-Abu), α,β-Dehydro-2-aminobutyric Acid (Δ-Abu), 1-Aminocyclopropane-1-carboxylic Acid (ACPC), Aminoisobutyric Acid (Aib), 2-Amino-thiazoline-4-carboxylic Acid, 5-Aminovaleric Acid (5-Ava), 6-Aminohexanoic Acid (6-Ahx), 8-Aminooctanoic Acid (8-Aoc), 11-Aminoundecanoic Acid (11-Aun), 12-Aminododecanoic Acid (12-Ado), 2-Aminobenzoic Acid (2-Abz), 3-Aminobenzoic Acid (3-Abz), 4-Aminobenzoic Acid (4-Abz), 4-Amino-3-hydroxy-6-methylheptanoic Acid (Statine, Sta), Aminooxyacetic Acid (Aoa), 2-Aminotetraline-2-carboxylic Acid (Atc), 4-Amino-5-cyclohexyl-3-hydroxypentanoic Acid (ACHPA), para-Aminophenylalanine (4-NH2-Phe), Biphenylalanine (Bip), para-Bromophenylalanine (4-Br-Phe), ortho-Chlorophenylalanine (2-Cl-Phe), meta-Chlorophenylalanine (3-Cl-Phe), para-Chlorophenylalanine (4-Cl-Phe), meta-Chlorotyrosine (3-Cl-Tyr), para-Benzoylphenylalanine (Bpa), tert-Butylglycine (Tle), Cyclohexylalanine (Cha), Cyclohexylglycine (Chg), 2,3-Diaminopropionic Acid (Dpr), 2,4-Diaminobutyric Acid (Dbu), 3,4-Dichlorophenylalanine (3,4-Cl2-Phe), 3,4-Diflurorphenylalanine (3,4-F2-Phe), 3,5-Diiodotyrosine (3,5-12-Tyr), ortho-Fluorophenylalanine (2-F-Phe), meta-Fluorophenylalanine (3-F-Phe), para-Fluorophenylalanine (4-F-Phe), meta-fluorotyrosine (3-F-Tyr), Homoserine (Hse), Homophenylalanine (Hfe), Homotyrosine (Htyr), 5-Hydroxytryptophan (5-OH-Trp), Hydroxyproline (Hyp), para-Iodophenylalanine (4-I-Phe), 3-lodotyrosine (3-I-Tyr), Indoline-2-carboxylic Acid (Idc), Isonipecotic Acid (Inp), meta-methyltyrosine (3-Me-Tyr), 1-Naphthylalanine (1-Nal), 2 Naphthylalanine (2-Nal), para-Nitrophenylalanine (4-NO2-Phe), 3-Nitrotyrosine (3-NO2-Tyr), Norleucine (Nle), Norvaline (Nva), Ornithine (Orn), ortho-Phosphotyrosine (H2PO3-Tyr), Octahydroindole-2-carboxylic Acid (Oic), Penicillamine (Pen), Pentafluorophenylalanine (F5-Phe), Phenylglycine (Phg), Pipecolic Acid (Pip), Propargylglycine (Pra), Pyroglutamic Acid (pGlu), Sarcosine (Sar), Tetrahydroisoquinoline-3-carboxylic Acid (Tic), and Thiazolidine-4-carboxylic Acid (Thioproline, Th). Stereochemistry of amino acids may be designated by preceding the name or abbreviation with the designation “D” or “d” or “L” or “l” as appropriate. Additionally, αN-alkylated amino acids may be employed, as well as amino acids having amine-containing side chains (such as Lys and Orn) in which the amine has been acylated or alkylated.

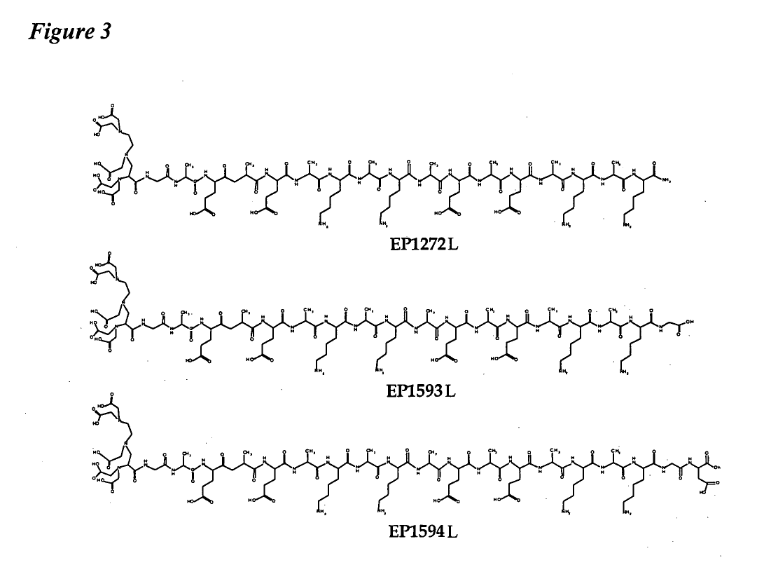

In some embodiments, the N- or C-terminus of an assembling peptide can be modified, e.g., to reduce overall aggregate size. Negatively charged moieties, for example, acidic groups, may be included at the C-terminus. Examples of acidic groups include linear diacids (e.g., having the structure HO2C(CH2)nCO2H, where when n can range from 1 to 8 (e.g., malonic, succinic, glutaric, adipic, pimelic, suberic, azelaic, sebacic acid, respectively)); aspartic acid, glutamic acid, and aromatic acids, such as phthalic, isophthalic, terephthalic acids. See FIG. 3. Examples of other negatively charged moieties include, but are not limited to, sulfates, nitrates, and phosphates.

Assembling peptides can associate with one another spontaneously (e.g., without crosslinking agents or covalent linkages) under appropriate conditions to form peptide aggregates. Assembling peptides that associate to form a peptide aggregate may have the same or different amino acid sequences. If an assembling peptide associates with an identical assembling peptide to form a peptide aggregate, it is referred to as a “self-assembling” peptide. Appropriate conditions for forming peptide aggregates involve considerations such as pH, temperature, solvent (e.g., buffer), and salt concentration and can be determined by one of skill in the art routinely. The ability of a peptide to associate with another peptide (which may have the same or a different amino acid sequence) to form a peptide aggregate can be evaluated by a variety of methods known to those of ordinary skill in the art, including spectroscopic assays (e.g., circular dichroism, NMR, light scattering assays); electrophoretic assays (e.g., mobility shift assays); centrifugation and filtration methods (e.g., ultrafiltration); sedimentation assays; and chromatographic (e.g. SEC-LS) assays.

In some embodiments, an assembling peptide associates with another assembling peptide having the same or substantially the same amino acid sequence over at least a portion of its length (e.g., at least 70%, at least 80%, at least 90%, or at least 95% of its length). An assembling peptide having substantially the same amino acid sequence can exhibit greater than 60%, greater than 70%, greater than 80%, greater than 90%, or greater than 95% sequence identity with another assembling peptide. Percent sequence identity can be calculated by determining the number of matched amino acids in aligned peptide sequences, dividing the number of matched amino acids by the total number of aligned amino acids, and multiplying by 100. A matched amino acid position refers to a position in which identical amino acids occur at the same position in aligned peptide sequences.

To determine percent sequence identity, a target peptide sequence can be compared to the identified peptide sequence using the BLAST 2 Sequences (Bl2seq) program from the stand-alone version of BLASTZ containing BLASTP version 2.0.14. This stand-alone version of BLASTZ can be obtained from Fish & Richardson's web site (www.fr.com/blast) or the U.S. government's National Center for Biotechnology Information web site (www.ncbi.nlm.nih.gov). Instructions explaining how to use the Bl2seq program can be found in the readme file accompanying BLASTZ. Bl2seq performs a comparison between two peptide sequences using the BLASTP algorithm. To compare two peptide sequences, the options of Bl2seq are set as follows: -i is set to a file containing the first peptide sequence to be compared (e.g., C:\seq1.txt); -j is set to a file containing the second peptide sequence to be compared (e.g., C:\seq2.txt); -p is set to blastp; -o is set to any desired file name (e.g., C:\output.txt); and all other options are left at their default setting. The following command will generate an output file containing a comparison between two peptide sequences: C:\Bl2seq-i c:\seq1.txt -j c:\seq2.txt -p blastp -o c:\output.txt. If the target sequence shares homology with any portion of the identified sequence, then the designated output file will present those regions of homology as aligned sequences. If the target sequence does not share homology with any portion of the identified sequence, then the designated output file will not present aligned sequences.

Once aligned, a length is determined by counting the number of consecutive amino acids from the target peptide sequence presented in alignment with amino acids from the identified peptide sequence starting with any matched position and ending with any other matched position. A matched position is any position where an identical amino acid is presented in both the target and identified sequence. Gaps presented in the target sequence are not counted since gaps are not amino acids. Likewise, gaps presented in the identified sequence are not counted since target sequence amino acids are counted, not amino acids from the identified sequence. The percent identity over a particular length is determined by counting the number of matched positions over that length and dividing that number by the length followed by multiplying the resulting value by 100.

Assembling peptides can associate to form peptide aggregates having a defined secondary and/or tertiary peptide structure. For example, peptide aggregates can exhibit structures such as beta sheet structures or alpha helices. Beta sheet structures may be parallel or antiparallel beta sheets. Alpha helices may associate in bundles, e.g., four helix bundles, in any relative orientation of the helices. Methods for assessing secondary and/or tertiary peptide structure are known to those of ordinary skill in the art and include assays such as CD spectroscopy and NMR spectroscopy.

In some embodiments, assembling peptides can be amphiphilic and may be comprised of alternating hydrophobic and hydrophilic amino acids, where the hydrophilic residues are designed to be complementary in charge pairing and/or hydrogen bonding. Such peptides can spontaneously assemble to form peptide aggregates having an antiparallel beta-sheet conformation with alternating hydrophobic/hydrophilic surfaces. In such peptide aggregates, the side chains of non-polar amino acids (e.g., alanine, valine, leucine, isoleucine, phenylalanine, tryptophan, and glycine) generally are oriented toward the hydrophobic surface while the side chains of polar amino acids (e.g., arginine, lysine histidine, aspartate, glutamate, asparagine, and glutamine) generally are oriented toward the hydrophilic surface. In some embodiments, the side chains of polar amino acids can make complementary ionic or hydrogen bonding pairs periodically along the hydrophilic surface.

In some embodiments of the invention, peptide aggregates can undergo a tertiary and/or secondary structural transition, e.g., from beta-sheets to alpha-helices with changes in pH, solvent, concentration, and/or temperature. Such a transition can influence aggregation and/or elimination from a subject. For example, under certain conditions, beta-sheet stranded aggregates can transition to alpha-helix or random coiled monomers, which can be excreted rapidly from a subject.

In some embodiments, assembling peptides are under initial conditions where they are not capable of associating to form a peptide aggregate, e.g., under initial conditions such as low or high pH; in the presence of denaturing or emulsifying agents, solvents, or detergents (e.g., urea, guanidine HCL, SDS); or the use of low concentrations of peptides. Conditions may then be changed to allow the assembling peptides to aggregate to form a peptide aggregate. For example, assembling peptides can be administered to a subject under initial conditions in which the assembling peptides cannot associate. After administration, however, the assembling peptides are capable of associating to form aggregates, e.g., because of a change in peptide concentration, pH, solvent, etc. once the assembling peptides are, for example, in the subject's bloodstream. Other possible initial conditions that prevent association of assembling peptides into aggregates include the use of surfactants or detergents in assembling peptide formulations; the use of low aqueous formulations, such as oil based formulations; the use of micelles to trap the assembling peptides prior to administration; and lyophilization of the assembling peptides with dilution just prior to administration.

In other embodiments, assembling peptides comprising target binding moieties having affinity for the same target can be administered to a subject under initial conditions that do not allow association, e.g., low assembling peptide concentrations or high denaturant concentration. Once administered to the subject, however, the assembling peptides can bind to the target and can associate in vivo, e.g., because of a higher local concentration at the target in vivo or because denaturant concentration has been reduced by injection and mixing with the subject's blood volume.

One example of a self-assembling peptide capable of forming peptide aggregates having an antiparallel beta-sheet conformation has the following amino acid sequence:

AEAEAKAKAEAEAKAK (SEQ ID NO: 1).

Additional examples of assembling peptides capable of forming peptide aggregates having beta sheet structures are:

| | |
|---|---|
| MDYEIKFH; | (SEQ ID NO: 2) |
| MDYNIQFH; | (SEQ ID NO: 3) |
| MDYKFKFN; | (SEQ ID NO: 4) |
| NFDLNLD; | (SEQ ID NO: 5) |
| EIQFEID; | (SEQ ID NO: 6) |
| NIDFQFD; | (SEQ ID NO: 7) |
| DLQLQIR; | (SEQ ID NO: 8) |
| DIEIEIR; | (SEQ ID NO: 9) |
| EVDIEIR; | (SEQ ID NO: 10) |
| RVQVHIH; | (SEQ ID NO: 11) |
| RVHIQLN; | (SEQ ID NO: 12) |
| RVHINLD; | (SEQ ID NO: 13) |
| KVDFHVN; | (SEQ ID NO: 14) |
| HIKVDFH; | (SEQ ID NO: 15) |
| QLKFHVN; | (SEQ ID NO: 16) |
| DVEVKMH; | (SEQ ID NO: 17) |
| ELQIDMH; | (SEQ ID NO: 18) |
| and | |
| EFNLKMH. | (SEQ ID NO: 19) |

Other assembling peptides can form peptide aggregates having structures containing one or more alpha helices, e.g., four helix bundles. Four helix bundle peptide aggregates may be formed from two peptide chains or from four peptide chains. Examples of assembling or self-assembling peptides capable of forming peptide aggregates having structures containing one or more helices include:

| | |
|---|---|
| DLENLLEKFEQLIK; | (SEQ ID NO: 20) |
| KLNHVVQELQELVQ; | (SEQ ID NO: 21) |
| KLKNLLNDFEDLIN; | (SEQ ID NO: 22) |
| NVQQLLKKLQQMIQ; | (SEQ ID NO: 23) |
| EIEDLLQKLQELME; | (SEQ ID NO: 24) |
| KIQKIIEKVNELMQ; | (SEQ ID NO: 25) |
| DLHNLINKLDDVMQ; | (SEQ ID NO: 26) |
| KMHDLIDDLHHLLN; | (SEQ ID NO: 27) |
| KLNDLLEDLQEVLK; | (SEQ ID NO: 28) |
| HLQNVIEDIHDFMQ; | (SEQ ID NO: 29) |
| KLQEMMKEFQQVLD; | (SEQ ID NO: 30) |
| and | |
| NIKEIFHHLEELVH. | (SEQ ID NO: 31) |

Assembling peptides that can associate to form structures having helices, e.g., four helix bundles, allow precise control of peptide aggregate size. In one embodiment, a four helix bundle formed from the association of four assembling peptides includes four metal binding moieties (e.g., one per assembling peptide) and one or more target binding moieties. Additional information on assembling peptides capable of forming beta sheets, helices, and helix bundles can be found in, e.g., Proc. Natl. Acad. Sci. USA 92:6349-6353 (1995) and Protein Science 12: 92-102 (2003).

Metal Binding Moieties

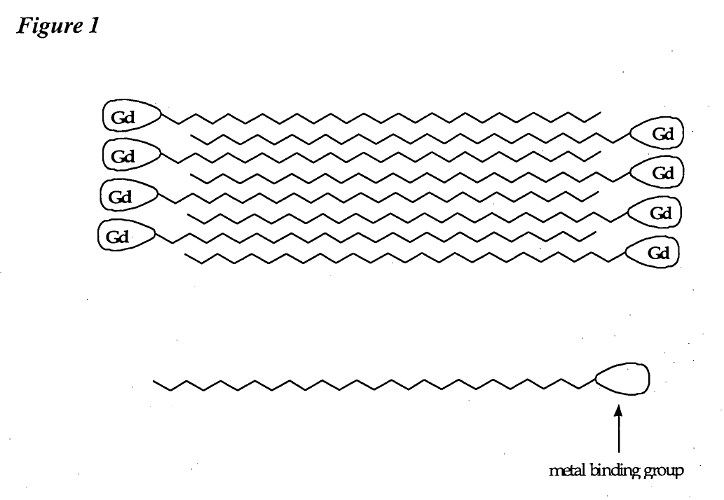

Assembling peptides can include a metal binding moiety, or a metal binding group, that is attached covalently to the peptide, either directly or via a linker (L). Metal binding moieties can be linked covalently (and optionally through a linker L) to the C terminus, N terminus, and/or amino acid side chains of an assembling peptide. Metal binding moieties bind metals, such as paramagnetic metal ions with atomic numbers 21-29, 42, 44, 57-83. Preferred paramagnetic metal ions are selected from the group consisting of Gd(III), Fe(III), Mn(II and III), Cr(III), Cu(II), Dy(III), Tb(III and IV), Ho(III), Er(III), Pr(III) and Eu(II and III). Gd(III) is particularly useful. Note that, as used herein, the term “Gd” is meant to convey the ionic form of the metal gadolinium; such an ionic form can be written as GD(III), GD3+, gado, etc., with no difference in ionic form contemplated. Peptide aggregates that include assembling peptides having metal binding moieties bound to paramagnetic metal ions can be useful as MR contrast agents (see FIG. 1).

Other metal binding moieties can bind radionuclides (e.g., Mn-51, Fe-52, Cu-60, Ga-68, As-72, Tc-94m, In-110, Y-90, Tc-99m, In-111, Sc-47, Ga-67; Cr-51, Sn-177m, Cu-67, Tm-167, Ru-97, Re-188, Lu-177, Au-199, Pb-203, or Ce-141). Peptide aggregates including assembling peptides containing metal binding moieties bound to radionuclides can be useful for diagnostic and/or therapeutic purposes.

Typically, a metal binding moiety is an organic chelating ligand. In these embodiments, an assembling peptide has a general formula: P-(C)n, where P refers to an assembling peptide amino acid sequence; C refers to an organic chelating ligand; and n can be 1 to 10. Exemplary organic chelating ligands include DOTA, DOTP, DO3A, DOTAGA, NOTA, and DTPA. When a chelating ligand is complexed with a metal ion, it can be referred to as a metal chelate. For MRI, metal chelates such as gadolinium diethylenetriaminepentaacetate (DTPA.Gd), gadolinium tetraamine 1,4,7,10-tetraazacyclododecane-N,N′,N″,N″′-tetraacetate (DOTA.Gd), gadolinium 1,4,7,10-tetraazacyclododecane- 1,4,7-triacetate (DO3A.Gd), and bb(CO)DTPA.Gd are particularly useful. In certain embodiments, DOTAGA may be preferred.

The C can be complexed to a paramagnetic metal ion, including Gd(III), Fe(III), Mn(II), Mn(III), Cr(III), Cu(II), Dy(III), Ho(III), Er(III), Pr(III), Eu(II), Eu(III), Tb(III), Tb(IV), Tm(III), and Yb(III). Additional information regarding C groups and synthetic methodologies for incorporating them into peptides can be found in WO 01/09188, WO 01/08712, and WO 03/011115.

Target Binding Moieties and Targets

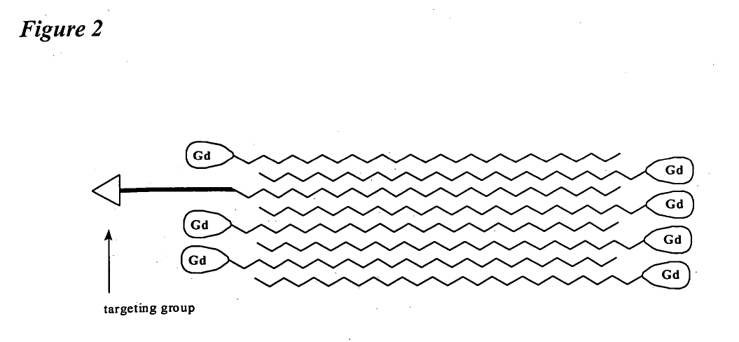

Assembling peptides also can include a target binding moiety that is attached covalently, either directly or via a linker, e.g., to the C-terminus, N-terminus, or the side chain of any amino acid in the peptide. For example, target binding moieties can be linked covalently to the C terminus, N terminus, and/or amino acid side chains (e.g., hydrophilic side chains) of a self-assembling peptide (FIG. 2). Peptide aggregates that include assembling peptides linked to a target binding moiety can exhibit an affinity for a particular target, (e.g., a biological target such as a polypeptide, enzyme, receptor, nucleic acid (e.g., DNA and RNA in all forms, including cDNA, genomic DNA, and mRNA), and tissue). Thus, peptide aggregates that include assembling peptides linked to a target binding moiety can be targeted to bind particular biological targets. Peptide aggregates can include assembling peptides linked to targeting moieties having affinities for different targets or that have affinities for different parts or components of a target.

A peptide aggregate in accord with the invention can include assembling peptides that are linked covalently to neither of, either of, or both of a metal binding moiety and a target binding moiety. For example, an assembling peptide can be linked to both a metal binding moiety and a target binding moiety. Alternatively, an assembling peptide can be linked only to a target binding moiety, or only to a metal binding moiety. See FIG. 2.

A target binding moiety can be any type of chemical compound, including small organic molecules and peptides. Peptides and small organic molecules can be screened for binding to a target by methods well known in the art, including equilibrium dialysis, affinity chromatography, and inhibition or displacement of probes known to bind to the target. Examples of useful target binding moieties are disclosed in WO 96/23526, WO 01/09188, and WO 03/011115. Example 8 below demonstrates the use of a phenylundecane moiety to target a peptide aggregate to HSA.

Targets for a peptide aggregate can be in any body compartment, cell, organ, or tissue or component thereof Preferred targets are those that are of diagnostic and therapeutic relevance, i.e., those that are associated with disease states. Particularly preferred targets are those in association with body fluids, and particularly those in association with blood, plasma, lymph and fluids of the central nervous system. Other preferred targets are proteins and receptors that either exist in high concentration or have a large number of binding sites for certain ligands. Included among such target proteins are enzymes and glycoproteins.

Human serum albumin (HSA), fibrin, and collagen are particularly useful targets. For vascular blood pool imaging, serum albumin is a preferred target. Since HSA is present at high concentration in serum (approximately 0.6 mM) and binds a wide array of molecules with reasonably high affinity, it is a preferred target plasma protein. HSA is a particularly preferred target for cardiovascular imaging; see WO 96/23526.

For imaging thrombi, fibrin is a preferred target because it is present in all thrombi and it can be targeted without interfering with the normal thrombolytic process. For additional details concerning target binding moieties that include fibrin-binding peptides, see PCT Patent Applications WO 01/09188 and WO 03/011115.

Other targets include, but are not limited to, alpha acid glycoprotein, fibrinogen, collagen, platelet GPIIb/IIIa receptor, chemotactic peptide receptor, somatostatin receptors, vasoactive intestinal peptides (VIP) receptor, bombesin/Gastrin release peptide receptor, integrin receptors, decorin, elastin, LOX-1, TLR (-2 and -4), CD36, SRAI/II, hyaluronic acid, LTB4, PAF, MCP-1, MAC-1, MMPs, CCR-1, CCR-3, LFA-1, Cathepsins, COX-1, COX-2, and TNF.

Linkers

In some embodiments, an assembling peptide is bound to a target binding moiety and/or a metal binding moiety through a linker (L). The L can include, for example, a linear, branched or cyclic peptide sequence. In one embodiment, a L can include a glycine residue or the linear dipeptide sequence G-G (glycine-glycine). In some embodiments, a L can cap the N-terminus of the peptide, the C-terminus, or both N- and C- termini, as an amide moiety. Other exemplary capping moieties include sulfonamides, ureas, thioureas and carbamates. A L can also include linear, branched, or cyclic alkanes, alkenes, or alkynes, or phosphodiester moieties. An L may be substituted with one or more functional groups, including ketone, ester, amide, ether, carbonate, sulfonamide, or carbamate functionalities. Specific Ls contemplated also include NH——CO—NH—; —CO—(CH₂)ₙ—NH—, where n=1 to 10; dpr; dab; —NH-Ph-; —NH—(CH₂)ₙ—, where n=1 to 10; —CO—NH—; —(CH₂)ₙ—NH—, where n=1 to 10; —CO—(CH₂)ₙ—NH—, where n=1 to 10; and —CS—NH—.

Additional examples of Ls and synthetic methodologies for incorporating them into peptides are set forth in WO 01/09188, WO 01/08712, and WO 03/011115.

Synthesis of Assembling Peptides Containing Metal Binding Moieties or Target Binding Moieties

Synthesis of peptides containing metal binding moieties may be carried out in the following manner. First, an assembling peptide can be synthesized with or without a C-terminal linker, typically using solid phase peptide synthesis. A C-terminal linker may be conveniently derived from the solid phase synthesis resin, and an N-terminus linker can be coupled to the peptide during the solid phase synthesis. Typically, metal chelating ligand moieties and/or target binding moieties are then coupled to the peptide. Protecting groups can be removed to provide the metal chelating ligand, and then metal chelates prepared by complexing metal ions. Radionuclide compounds of this invention can be prepared from ligands using commercially available radionuclides (for example, ^{99m}Tc from Nycomed Amersham Boston cat. #RX-290195, ¹¹¹In from NEN Life Science Products cat. # NEZ304, or ¹⁵³Gd from NEN Life Science Products cat. # NEZ142) by reaction in aqueous media, typically at pH 4-6 for 1 hour.

Properties of Peptide Aggregates

Peptide aggregates of the invention can bind a target, such as collagen, HSA, or fibrin. For example, at least 10% (e.g., at least 30%, 40%, 50%, 70%, 80%, 90%, 92%, 94%, or 96%) of the aggregate can be bound to the desired target at physiologically relevant concentrations. The extent of binding to a target can be assessed by a variety of equilibrium binding methods, e.g., ultrafiltration methods; equilibrium dialysis; affinity chromatography; or competitive binding inhibition or displacement of probe compounds.

Peptide aggregates containing metal binding moieties that are used as MR contrast agents can exhibit high relaxivity as a result of target binding (e.g., to HSA, collagen, or fibrin), which can lead to better image resolution. The increase in relaxivity upon binding is typically 1.5-fold or more (e.g., at least a 2, 3, 4, 5, 6, 7, 8, 9, or 10 fold increase in relaxivity). Targeted aggregates having 7-8 fold, 9-10 fold, or greater than 10 fold increases in relaxivity are particularly useful. Typically, relaxivity is measured using an NMR spectrometer. A particularly useful relaxivity at 20 MHz and 37° C. is at least 5 mM⁻¹s⁻¹ per paramagnetic metal ion (e.g., at least 8, 10, 15, 20, 25, 30, 35, 40, or 60 mM⁻¹s⁻¹ per paramagnetic metal ion). Aggregates having a relaxivity greater than 20 mM⁻¹s⁻¹, or greater than 35 mM⁻¹s⁻¹, at 20 MHz and 37° C. are particularly useful.

Use of Peptide Aggregates as Contrast Agents

Peptide aggregates containing assembling peptides having metal binding moieties can be used as MRI contrast agents and can typically be used in the same manner as conventional MRI contrast agents. Typically, the contrast agent is administered to a patient (e.g., an mammal, such as a human) and an MR image of the patient is acquired. Generally, the clinician will acquire an image of an area containing the target of interest. For example, the clinician may acquire an image of the heart if the contrast agent targets collagen. The clinician may acquire one or more images at a time before, during, or after administration of the contrast agent.

Certain MR techniques and pulse sequences may be preferred in the methods of the invention. For example, the MR images can be steady state MR images. Examples of desirable pulse sequences include cardiac gated 2d spin echo (TE/TR=15/1RR) sequences, T₁ weighted spoiled echo gradient sequences (cardiac gated, flip/TE/TR=30°/2/8), IR-prepped gradient echo sequences, and navigated IR-prepped sequences.

In some embodiments, a contrast-enhancing imaging sequence that preferentially increases a contrast ratio of a magnetic resonance signal of the target having a contrast agent bound thereto relative to the magnetic resonance signal of background or flowing blood is used. These techniques include, but are not limited to, black blood angiography sequences that seek to make blood dark, such as fast spin echo sequences; flow-spoiled gradient echo sequences; and out-of-volume suppression techniques to suppress in-flowing blood. These methods also include flow independent techniques that enhance the difference in contrast due to the T1 difference between contrast-enhanced target and blood and tissue, such as inversion-recovery prepared or saturation-recovery prepared sequences that will increase the contrast between the target and background tissues. Methods of preparation for T2 techniques may also prove useful. Finally, preparations for magnetization transfer techniques may also improve contrast with contrast agents of the invention.

Methods may be used that involve the acquisition and/or comparison of contrast-enhanced and non-contrast images and/or the use of one or more additional contrast agents. For example, methods as set forth in U.S. patent application Ser. No. 09/778,585, entitled MAGNETIC RESONANCE ANGIOGRAPHY DATA, filed Feb. 7, 2001 and U.S. patent application Ser. No. 10/209,416, entitled SYSTEMS AND METHODS FOR TARGETED MAGNETIC RESONANCE IMAGING OF THE VASCULAR SYSTEM, filed Jul. 30, 2002 may be used.

Pharmaceutical Compositions

Contrast agents, peptide aggregates, and assembling peptides can be formulated as a pharmaceutical composition in accordance with routine procedures. As used herein, the pharmaceutical compositions of the invention can include pharmaceutically acceptable derivatives of the contrast agents, peptide aggregates, or assembling peptides. “Pharmaceutically acceptable” means that the agent can be administered to an animal without unacceptable adverse effects. A “pharmaceutically acceptable derivative” means any pharmaceutically acceptable salt, ester, salt of an ester, or other derivative of a contrast agent, aggregate, or peptide of this invention that, upon administration to a recipient, is capable of providing (directly or indirectly) a contrast agent, aggregate, or peptide of this invention or an active metabolite or residue thereof. Other derivatives are those that increase the bioavailability when administered to a mammal (e.g., by allowing an orally administered compound to be more readily absorbed into the blood) or which enhance delivery of the parent compound to a biological compartment (e.g., the brain or lymphatic system) thereby increasing the exposure relative to the parent species. Pharmaceutically acceptable salts of the contrast agents, aggregates, or peptides of this invention include counter ions derived from pharmaceutically acceptable inorganic and organic acids and bases known in the art.

Pharmaceutical compositions of the invention can be administered by any route, including both oral and parenteral administration. Parenteral administration includes, but is not limited to, subcutaneous, intravenous, intraarterial, interstitial, intrathecal, and intracavity administration. When administration is intravenous, phannaceutical compositions may be given as a bolus, as two or more doses separated in time, or as a constant or non-linear flow infusion. Thus, pharmaceutical compositions of the invention can be formulated for any route of administration.

Typically, pharmaceutical compositions for intravenous administration are solutions in sterile isotonic aqueous buffer. Where necessary, the composition may also include a solubilizing agent, a stabilizing agent, and a local anesthetic such as lidocaine to ease pain at the site of the injection. Generally, the ingredients will be supplied either separately, e.g. in a kit, or mixed together in a unit dosage form, for example, as a dry lyophilized powder or water-free concentrate. The composition may be stored in a hermetically sealed container such as an ampule or sachette indicating the quantity of active agent in activity units. Where the composition is administered by infusion, it can be dispensed with an infusion bottle containing sterile pharmaceutical grade “water for injection,” saline, or other suitable intravenous fluids. Where the composition is to be administered by injection, an ampule of sterile water for injection or saline may be provided so that the ingredients may be mixed prior to administration.

A contrast agent of the invention is preferably administered to the patient in the form of an injectable composition. The method of administering a contrast agent is preferably parenterally, meaning intravenously, intra-arterially, intrathecally, interstitially or intracavitarilly. Pharmaceutical compositions of this invention can be administered to mammals including humans in a manner similar to other diagnostic or therapeutic agents. The dosage to be admininistered, and the mode of administration will depend on a variety of factors including age, weight, sex, condition of the patient and genetic factors, and will ultimately be decided by medical personnel subsequent to experimental determinations of varying dosage followed by imaging as described herein. In general, dosage, required for diagnostic sensitivity or therapeutic efficacy will range from about 0.001 to 50,000 μg/kg, preferably between 0.01 to 25.0 μg/kg of host body mass. The optimal dose can be determined empirically.

The invention will be further described in the following examples, which do not limit the scope of the invention described in the claims.

### EXAMPLES

### Example 1

### Synthesis of EP1272L

This example demonstrates the synthesis of a self-assembling peptide having a covalently attached metal binding moiety. A self-assembling peptide having the sequence AEAEAKAKAEAEAKAK (SEQ ID NO: 1) was chemically modified to include a metal binding moiety (an organic chelating ligand) via a glycine linker to the N-terminus of the peptide; see EP1272L in FIG. 3. The EP1272L was prepared by SynPep, with the bbDTPA provided by EPIX. The compound was synthesized on a solid support using standard methods. HPLC purity 90%. MS data (M+H⁺)/1=2092, (M+2H⁺)/2=1046.5, (M+3H⁺)/3=698.

### Example 2

### EP1272L Complexed to Gd(III)

This example demonstrates the complexing of EP1272L with Gd(III). EP1272L (0.0842 g) was suspended in 2 mL water. To this were added 160 μL 1N NaOH, bringing the pH of the solution to 7.5. The volume was taken to 5 mL with addition of water and the solution gently swirled until all solids were dissolved. The chelatables were monitored by titration using xylenol orange as an indicator for free gadolinium. Following titration, 86.4 μL of 241 mM stock solution of GdCl₃ was added to the solution with stirring followed by 60 μL 1N NaOH. Low mass impurities were removed from the solution by centrifugation over a 0.02 μm anapore membrane (Vecta-Spin). This was followed by resuspension in 20 mL of water and filtration over a 5 μm (Durapore, low protein binding) syringe filter frit. At concentrations greater than 0.5 mM, EP1272L became a non-flowing viscous gel.

### Example 3

### Synthesis of EP1593L

This example demonstrates the synthesis of a self-assembling peptide having a covalently attached metal binding moiety and an acidic group (FIG. 3). EP1593L was prepared by SynPep, with the bbDTPA provided by EPIX. The compound was synthesized on a solid support using standard methods. HPLC purity 85%. MS data (M+2H⁺)/2=1075.9, (M+3H⁺)/3=717.5.

### Example 4

### EP1593L complexed to Gd(III)

This example demonstrates complexing of EP1593L with Gd(III). EP1593L (0.0336 g) was suspended in 4 mL water. To this were added 45 μL 1N NaOH, bringing the pH of the solution to neutral. This solution was filtered over a 5 μm (Durapore, low protein binding) syringe filter frit. To 2 mL of this solution was added 207 μL of a GdCl₃ solution (19.3 mM stock). The solution was gently swirled and then monitored for excess gadolinium using xylenol orange as an indicator. Aliquots of the EP1593L solution were then added and monitored until no free gadolinium remained. The solution was neutralized again using 1N NaOH.

### Example 5

### Synthesis of EP1594L

This example demonstrates the synthesis of a self-assembling peptide having a covalently attached metal binding moiety and an acidic group. EP1594L (FIG. 3) was prepared by SynPep, with the bbDTPA provided by EPIX. The compound was synthesized on a solid support using standard methods. HPLC purity 85%. MS data (M+H⁺)/1=2266.9, (M+2H⁺)/2=1133.1, (M+3H⁺)/3=756.1.

### Example 6

### EP1594L complexed to Gd(III)

This example demonstrates the complexing of EP1594L with Gd(III). EP1594L (0.0332 g) was suspended in 3 mL water. To this were added 45 μL 1N NaOH, bringing the pH of the solution to neutral. This solution was filtered over a 5 μm (Durapore, low protein binding) syringe filter frit. To 1.5 mL of this solution was added 186 μL of a GdCl₃ solution (19.3 mM stock). The solution was gently swirled and then monitored for excess gadolinium using xylenol orange as an indicator. Aliquots of the GdCl₃ stock solution were added and monitored until free gadolinium was observed. This was followed by additional EP1594L stock solution to remove the excess gadolinium. The solution was neutralized again using 1N NaOH.

### Example 7

### Synthesis of EP1595

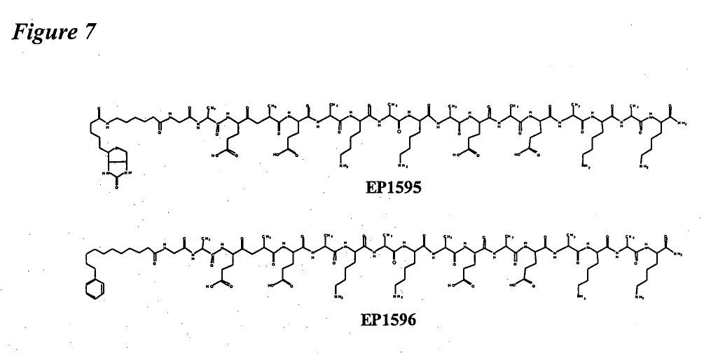

This example demonstrates the synthesis of a self-assembling peptide containing a target binding moiety. EP1595 (FIG. 7) was prepared by SynPep, using commercially available biotin. The compound was synthesized on a solid support using standard methods. HPLC purity 91.4%. MS data (M+H⁺)/1=2013, (M+2H⁺)/2=1006.9, (M+3H⁺)/3=671.5, (M+4H⁺)/4=503.95.

### Example 8

### Synthesis of EP1596

This example demonstrates the synthesis of a self-assembling peptide containing a target binding moiety. EP1596 (FIG. 7) was prepared by SynPep, using commercially available phenylundecanoic acid. The compound was synthesized on a solid support using standard methods. HPLC purity 98.8%. MS data (M+H⁺)/1=1917, (M+2H⁺)/2=959.3, (M+3H⁺)/3=639.9. EP1596 can be used to target human serum albumin (HSA).

### Example 9

### Targeted Metallopeptide Aggregates

As described in Examples 1-8, a self-assembling peptide having the sequence AEAEAKAKAEAEAKAK (SEQ ID NO: 1) was chemically modified to include a metal binding moiety (an organic chelating ligand) via a glycine linker to the N-terminus of the peptide; see EP1272L in FIG. 3. Modified self-assembling peptides having the same metal binding moiety at the N-terminus and further including acidic moieties at the C-terminus of the peptide were also made (see EP 1593L and EP1594L in FIG. 3). The peptide alone (e.g., unbound to the metal binding moiety) tended to form macroscopic membranes. The modified peptides (EP1272L, EP1593L, and EP1594L) tended to aggregate, but did not form macroscopic membranes.

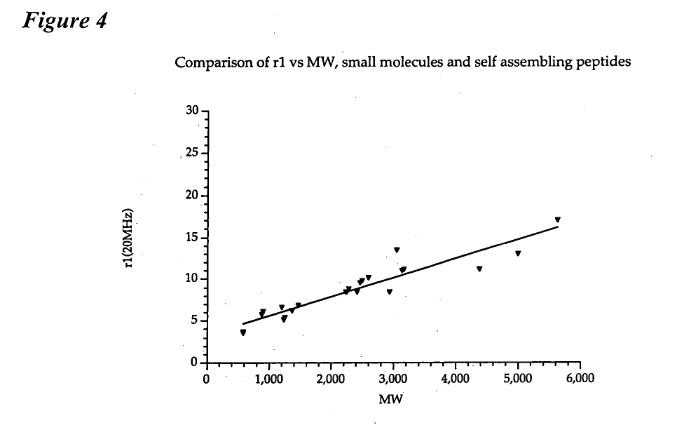

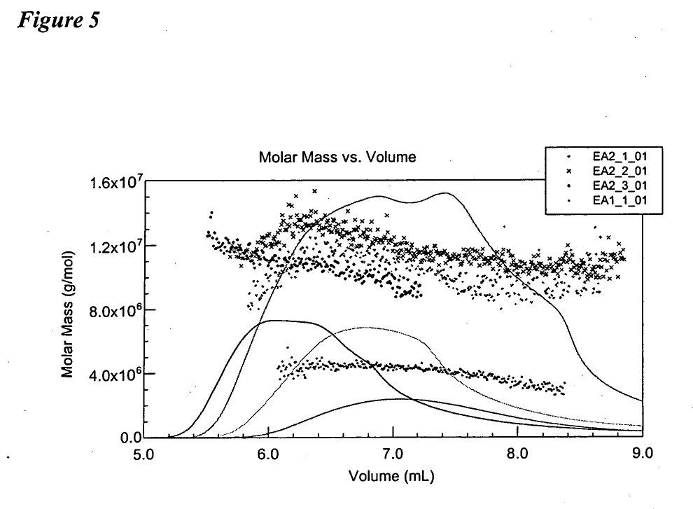

EP1272L peptide aggregation was demonstrated several ways. First, solutions of EP 1272L (FW=2,243 Da) were concentrated and purified by centrifugation in a filter device with a nominal molecular weight limit of 300,000 Da. The formation of high molecular weight aggregates allowed EP1272L to be retained by the filter device. Second, EP1272L relaxivity was measured in phosphate buffered saline (PBS) and found to be 25 mM⁻¹s⁻¹ at 0.47 T and 21 mM⁻¹s⁻¹ at 1.5 T. This relaxivity is significantly greater than would be expected based on the molecular weight of a EP1272 monomer (ee FIG. 4), suggesting that the monomer self assembles into higher molecular weight aggregates. Third, SEC-LS column retention and light scattering measurements of EP1272L were consistent with a large species having a mass distribution of 4×10⁶ to 13×10⁶ Da, and a hydrodynamic radius in the range of 155 nm to 210 nm. The SEC-LS measurement suggest that EP1272L forms aggregates consisting of approximately 2,000 to 6,000 monomers (FIG. 5).

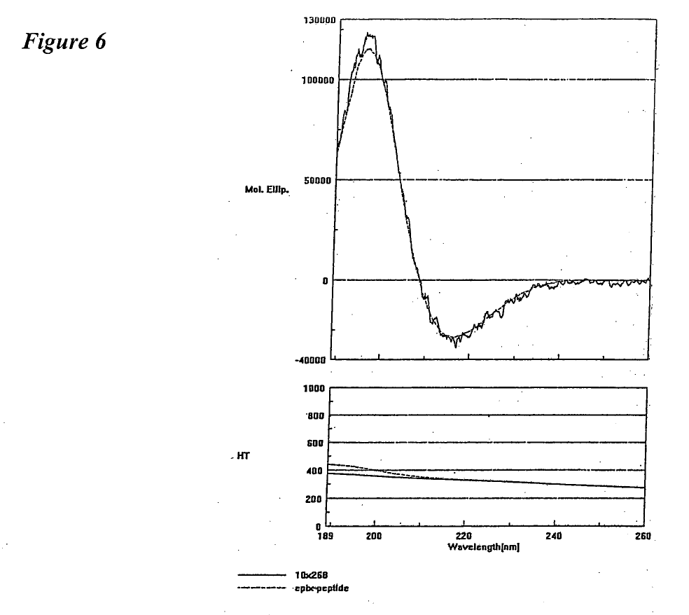

EP1272L aggregates were analyzed with circular dichroism (CD) spectroscopy. CD analysis of a 50 μM solution of EP1272L indicated the presence of beta-sheet structure (FIG. 6). This is consistent with previous reports for the AEAEAKAKAEAEAKAK peptide alone, which reportedly organizes into antiparallel beta sheets.

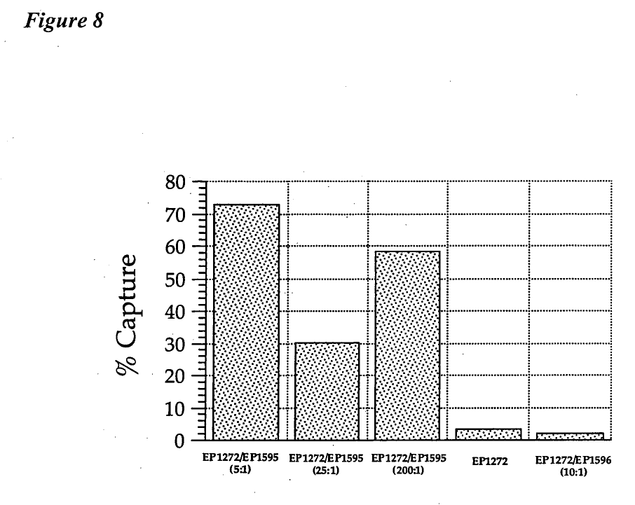

Two self-assembling peptides comprising target binding moieties were prepared (FIG. 7). EP1595 contained a C-terminal biotin target binding moiety; EP 1596 contained a C-terminal aromatic target binding moiety (phenylundecane). Formulation of EP1272L with biotinylated EP1595 yielded aggregates having affinity for streptavidin beads. EP1272L aggregates alone and EP1272L formulated with the phenylundecane peptide, EP1596, displayed substantially less affinity for streptavidin beads (FIG. 8). These results demonstrated the ability of the peptide aggregates to be targeted.

### Example 10

### Relaxivity Measurements

Relaxivity measurements were made in PBS. A relaxivity titration consisting of sequential additions of EP1272L (complexed to Gd(III)) to a tube containing approximately 0.6 mL of PBS was performed. Following each addition of EP1272, the sample was thoroughly mixed by manual agitation and the temperature allowed to equilibrate for 10 minutes at 37±2° C. prior to T1 determination. The longitudinal (T1) relaxation time was determined by use of the inversion recovery pulse sequence and fitting the intensity data to an exponential decay. Measurements at 20 MHz were made using a Bruker Minispec PC 120/125/Vts nuclear magnetic resonance process analyzer. The temperature was regulated with a Haake D8-GH circulating bath at 37±2° C. Measurements at 64.5 MHz were made using a Varian XL300 adjusted to a field strength of 1.5 T with a broad band variable temperature probe tuned to 64.5 MHz. The relaxivity of EP1272 at 0.5 T and 1.5 T were 24.9 mM⁻¹s⁻¹ and 21.0 mM⁻¹s⁻¹ respectively. The relaxivity of EP1593 at 0.5 T was assessed in a similar manner and was 19.9 mM⁻¹s⁻¹. The relaxivity of EP1594 was assessed in a similar manner at 0.5 T and was 22.0 mM⁻¹s⁻¹.

### Example 11

### Size Distribution Measurements

The size distribution of some aggregates were determined by HPLC size exclusion chromatography and laser light scattering (SEC-LS). In several runs, EP1272L produced a single peak by gel filtration HPLC. The molecular weight averages ranged from 4×10⁶ to 13×10⁶ Da, depending upon the concentration of EP1272 eluting from the column. The average hydrodynamic radii of the aggregates ranged from 155 nm to 210 nm, again depending upon the concentration of EP1272 eluting from the column.

### Example 12

### CD Spectrum of EP1272L

Ultraviolet circular dichroism studies were carried out by Alliance Protein Labs using a Jasco J-715 spectropolarimeter. UV CD studies of EP1272L at 50 μM and 5 μM peptide were indistinguishable. The spectra were characterized by a negative peak at 216 nm and a positive peak at 196 nm. These characteristics are typical of a beta sheet structure.

### Example 13

### Formulation of Avidin-Targeted Aggregates of EP 1272L

EP1272L aggregates were targeted to avidin by incorporation of a biotinylated peptide (e.g., an assembling peptide containing a target binding moiety (EP1595)) into the aggregates. To 70 μL of a 1.54 mM stock solution of EP1272L was added varying amounts of EP1595. The pH was then reduced to 1.5 by addition of 1N HCl to induce mixing of the peptides. The solution was neutralized by addition of 1N NaOH and the volume brought to 500 μL with addition of water. Aggregates containing 20% biotinylated peptide (5:1 ratio of EP1272 to EP1595), 4% biotinylated-peptide (25:1 ratio), and 0.5% biotinylated peptide (200:1) were prepared.

### Example 14

### Formulation of EP1596 with EP 1272L

EP1272L aggregates containing the phenylundecane substituted peptide, EP1596, in a 10:1 ratio were prepared as described above.

### Example 15

### Avidin Affinity Assay

The three EP1595/EP1272 formulations, one EP1596/EP1272 formulation, and an EP1272 formulation were assayed for affinity to streptavidin magnetic beads. Each sample was diluted so as to maintain a constant ratio of biotinylated peptide and streptavidin binding sites in the assay. Solutions without biotin were treated as though they contained 0.5% EP1595. After several rinses the magnetic beads were digested in acid. The extent of capture was quantified by gadolinium ICP. See FIG. 8.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A peptide aggregate comprising two or more assembling peptides, wherein at least one of said assembling peptides comprises a metal binding moiety.

2. The aggregate of claim 1, wherein the hydrodynamic radius of said aggregate is 2 nm to 500 nm.

3. The aggregate of claim 1, wherein at least one of said assembling peptides comprises a target binding moiety having an affinity for a target.

4. The aggregate of claim 3, wherein said target is selected from the group consisting of HSA, fibrin, collagen, decorin, and elastin.

5. The aggregate of claim 1, wherein said metal binding moiety is an organic chelating ligand.

6. The aggregate of claim 5, wherein said organic chelating ligand is selected from the group consisting of DTPA, DOTA, DOTP, DO3A, DOTAGA, and NOTA.

7. The aggregate of claim 1, wherein said two or more assembling peptides independently comprise an amino acid sequence selected from the group consisting of:

| | |
|---|---|
| AEAEAKAKAEAEAKAK; | (SEQ ID NO: 1) |
| MDYEIKFH; | (SEQ ID NO: 2) |
| MDYNIQFH; | (SEQ ID NO: 3) |
| MDYKFKFN; | (SEQ ID NO: 4) |
| NFDLNLD; | (SEQ ID NO: 5) |
| EIQFEID; | (SEQ ID NO: 6) |
| NIDFQFD; | (SEQ ID NO: 7) |
| DLQLQIR; | (SEQ ID NO: 8) |
| DIEIEIR; | (SEQ ID NO: 9) |
| EVDIEIR; | (SEQ ID NO: 10) |
| RVQVHIH; | (SEQ ID NO: 11) |
| RVHIQLN; | (SEQ ID NO: 12) |
| RVHINLD; | (SEQ ID NO: 13) |
| KVDFHVN; | (SEQ ID NO: 14) |
| HIKVDFH; | (SEQ ID NO: 15) |
| QLKFHVN; | (SEQ ID NO: 16) |
| DVEVKMH; | (SEQ ID NO: 17) |
| ELQIDMH; | (SEQ ID NO: 18) |
| EFNLKMH; | (SEQ ID NO: 19) |
| DLENLLEKFEQLIK; | (SEQ ID NO: 20) |
| KLNHVVQELQELVQ; | (SEQ ID NO: 21) |
| KLKNLLNDFEDLIN; | (SEQ ID NO: 22) |
| NVQQLLKKLQQMIQ; | (SEQ ID NO: 23) |
| EIEDLLQKLQELME; | (SEQ ID NO: 24) |
| KIQKIIEKVNELMQ; | (SEQ ID NO: 25) |
| DLHNLINKLDDVMQ; | (SEQ ID NO: 26) |
| KMHDLIDDLHHLLN; | (SEQ ID NO: 27) |
| KLNDLLEDLQEVLK; | (SEQ ID NO: 28) |
| HLQNVIEDIHDFMQ; | (SEQ ID NO: 29) |
| KLQEMMKEFQQVLD; | (SEQ ID NO: 30) |
| and | |
| NIKEIFHHLEELVH. | (SEQ ID NO: 31) |

8. The aggregate of claim 5, wherein said organic chelating ligand is bound to a paramagnetic metal ion.

9. The aggregate of claim 8, wherein said paramagnetic metal ion is Gd (III).

10. The aggregate of claim 1, wherein said assembling peptides are self-assembling peptides.

11. The aggregate of claim 7, wherein said sequence is modified at the C-terminus by an acidic moiety.

12. The aggregate of claim 11, wherein said acidic moiety is selected from the group consisting of a linear diacid, glutamic acid, aspartic acid, phthalic acid, isophthalic acid, and terephthalic acid.

13. An assembling peptide having a structure: P—(C)n, wherein P refers to an assembling peptide amino acid sequence, C refers to an organic chelating ligand, and n can be 1 to 10.

14. The assembling peptide of claim 13, further comprising a target binding moiety.

15. An assembling peptide comprising a target binding moiety.

16. An assembling peptide comprising an amino acid sequence selected from the group consisting of:

| | |
|---|---|
| AEAEAKAKAEAEAKAK; | (SEQ ID NO: 1) |
| MDYEIKFH; | (SEQ ID NO: 2) |
| MDYNIQFH; | (SEQ ID NO: 3) |
| MDYKFKFN; | (SEQ ID NO: 4) |
| NFDLNLD; | (SEQ ID NO: 5) |
| EIQFEID; | (SEQ ID NO: 6) |
| NIDFQFD; | (SEQ ID NO: 7) |
| DLQLQIR; | (SEQ ID NO: 8) |
| DIEIEIR; | (SEQ ID NO: 9) |
| EVDIEIR; | (SEQ ID NO: 10) |
| RVQVHIH; | (SEQ ID NO: 11) |
| RVHIQLN; | (SEQ ID NO: 12) |
| RVHINLD; | (SEQ ID NO: 13) |
| KVDFHVN; | (SEQ ID NO: 14) |
| HIKVDFH; | (SEQ ID NO: 15) |
| QLKFHVN; | (SEQ ID NO: 16) |
| DVEVKMH; | (SEQ ID NO: 17) |
| ELQIDMH; | (SEQ ID NO: 18) |
| EFNLKMH; | (SEQ ID NO: 19) |
| DLENLLEKFEQLIK; | (SEQ ID NO: 20) |
| KLNHVVQELQELVQ; | (SEQ ID NO: 21) |
| KLKNLLNDFEDLIN; | (SEQ ID NO: 22) |
| NVQQLLKKLQQMIQ; | (SEQ ID NO: 23) |
| EIEDLLQKLQELME; | (SEQ ID NO: 24) |
| KIQKIIEKVNELMQ; | (SEQ ID NO: 25) |
| DLHNLINKLDDVMQ; | (SEQ ID NO: 26) |
| KMHDLIDDLHHLLN; | (SEQ ID NO: 27) |
| KLNDLLEDLQEVLK; | (SEQ ID NO: 28) |
| HLQNVIEDIHDFMQ; | (SEQ ID NO: 29) |
| KLQEMMKEFQQVLD; | (SEQ ID NO: 30) |
| and | |
| NIKEIFHHLEELVH; | (SEQ ID NO: 31) |

wherein said assembling peptide further comprises a target binding moiety, a metal binding moiety, or both.

17. An assembling peptide selected from the group consisting of EP1272L, EP1593L, EP1594L, EP1595, and EP1596.

18. A method for MR imaging of a target comprising:
i) administering to a patient a peptide aggregate, said peptide aggregate comprising at least one assembling peptide comprising a metal binding moiety bound to a paramagnetic metal ion and at least one assembling peptide comprising a target binding moiety having an affinity for said target; and
ii) subjecting said patient to MR imaging.

19. A method for MR imaging comprising:
i) administering to a patient a peptide aggregate, said peptide aggregate comprising at least one assembling peptide comprising a metal binding moiety bound to a paramagnetic metal ion; and
ii) subjecting said patient to MR imaging.

20. A method for MR imaging of a target, comprising:
i) administering to a patient at least two assembling peptides, said at least two assembling peptides capable of forming a peptide aggregate after administration to said patient, wherein at least one of said two assembling peptides comprises a metal binding moiety bound to a paramagnetic metal ion and at least one of said two assembling peptides comprises a target binding moiety having an affinity for said target; and
ii) subjecting said patient to MR imaging.
